(19) United States
(12) Patent Application Publication
(10) Pub. No.: US 2024/0358485 A1
(43) Pub. Date: Oct. 31, 2024

(54) ANIMAL DRUG DELIVERY SYSTEM AND METHOD

(71) Applicant: The Trustees of Indiana University, Bloomington, IN (US)

(72) Inventors: Brian D. Overshiner, McCordsville, IN (US); William Roy Thompson, Zionsville, IN (US); Parker Evans, Pittsburgh, PA (US); Perla Reyes Fernandez, Bloomington, IN (US); Daniel Georgiev Simeonov, Elkhart, IN (US)

(21) Appl. No.: 18/649,689

(22) Filed: Apr. 29, 2024

Related U.S. Application Data
(60) Provisional application No. 63/498,720, filed on Apr. 27, 2023.

(51) Int. Cl. A61D 7/00; A61M 15/00

(52) U.S. Cl. CPC A61D 7/00; A61M 15/0066; A61M 2205/3306; A61M 2205/581; A61M 2205/8206

(57) ABSTRACT

Animal drug delivery systems and methods. Such a system includes a carrier cartridge to carry and dispense a plurality of drug doses, a monitoring device, and a controller operatively coupled with the carrier cartridge and the monitoring device. The controller operates the carrier cartridge to dispense at least one of the drug doses to an animal subject at each of a plurality of preselected dispensing intervals. The controller is also configured to operate the monitoring device to record the animal subject ingesting the dispensed drug doses. The system may be configured to provide drug pellets to mice based on researcher needs, allow for mouse monitoring to confirm drug delivery, enable behavioral studies to be conducted using operant condition, and/or to fit into a specific lab research animal cage.

### CROSS-REFERENCE TO RELATED APPLICATIONS

This application claims the benefit of provisional U.S. Patent Application No. 63/498,720 filed Apr. 27, 2023, the contents of which are incorporated herein by reference.

### BACKGROUND OF THE INVENTION

The invention generally relates to animal drug delivery systems and methods of delivering a drug dose to an animal subject.

Rodents and various other animals are used for pharmaceutical, biochemical, and behavioral research. Often this research requires scores of rodent trials that span multiple months. Unfortunately, long term rodent drug and microbiology trials are time consuming and labor intensive. Frequently, such trials require a laboratory technician or a researcher to spend at least three hours a day providing the tested substance into the animal test subject, such as a rodent, either through oral delivery or injection delivery. This process can be both traumatic on the animal test subject and time consuming for the researcher.

While there are some existing automated drug delivery systems that attempt to mitigate the above-noted burdens on both the researchers and rodent test subjects, existing automated drug delivery systems typically have multiple limitations. For example, existing automated drug delivery systems typically do not allow for the researcher to regulate the drug delivery schedule, cannot monitor the test subjects, and/or cannot train the test subjects to ingest the drug pellet. Other limitations of many automated drug delivery systems include their inability to fit various different types of cages and/or their requirement for specialized cabinets. For example, a particular commercially-available drug delivery system used in research lacks any features for monitoring whether the rodent actually ingests a particular dose of the drug or for controlling drug dosing, such as when, how often, and/or how much drug the animal test subject receives. Commercially-available drug delivery systems may not be sized or otherwise configured for use with commonly used cage base configurations for mouse cages, such as the “Max 75” cage base.

Therefore, it would be desirable to have a way to automate drug delivery to rodents to shorten the time spent by researchers on daily manual drug injections to rodents and still be able to monitor and/or control the amount of drug the animal test subject is actually dosed.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, an automated drug delivery system and/or a method of delivering a drug dose to an animal subject.

According to a nonlimiting aspect, an automated drug delivery system includes a carrier cartridge configured to carry and dispense a plurality of drug doses, a monitoring device, and a controller operatively coupled with the carrier cartridge and the monitoring device. The controller is configured to operate the carrier cartridge to dispense at least one of the plurality of drug doses to an animal subject at each of a plurality of preselected dispensing intervals. The controller is also configured to operate the monitoring device to record whether an animal subject ingests each drug dose dispensed by the carrier cartridge.

According to another nonlimiting aspect, a method of automatically delivering a drug dose to an animal subject is provided. The method uses an automated drug delivery system comprising a carrier cartridge configured to carry and dispense a plurality of drug doses, a monitoring device, and a controller operatively coupled with the carrier cartridge and the monitoring device. The method includes operating the carrier cartridge with the controller to dispense at least one of the plurality of drug doses to the animal subject at each of a plurality of preselected dispensing intervals, and operating the monitoring device with the controller to record whether the animal subject ingests each drug dose dispensed by the carrier cartridge.

Technical aspects of automated drug delivery systems and methods having features as described above preferably include the ability to facilitate drug delivery without having to change existing cages, and/or the capability for reducing skin degradation and trauma on animal test subjects that can occur with subjects that receive multiple daily injections.

These and other aspects, arrangements, features, and/or technical effects will become apparent upon detailed inspection of the figures and the following description.

### BRIEF DESCRIPTION OF THE DRAWINGS

FIG. 1 is an isometric view of an automated drug delivery system according to certain nonlimiting aspects of the present invention.

FIG. 2 is an isometric view of an interior portion of the automated drug delivery system of FIG. 1 and control components disposed within the interior portion.

FIG. 3 represents some of the control components of FIG. 2 removed from the interior portion of the automated drug delivery system of FIG. 1.

FIG. 4 is a perspective view of the automated drug delivery system operationally disposed inside a Max 75 cage base.

FIG. 5 illustrates certain steps in a nonlimiting example of using the automated drug delivery system during an experiment.

### DETAILED DESCRIPTION OF THE INVENTION

The intended purpose of the following detailed description of the invention and the phraseology and terminology employed therein is to describe what is shown in the drawings, which relate to one or more nonlimiting embodiments of the invention, and to describe certain but not all aspects of what is depicted in the drawings, including the embodiment(s) to which the drawings relate. The following detailed description also identifies certain but not all alternatives of the embodiment(s) depicted in the drawings. As nonlimiting examples, the invention encompasses additional or alternative embodiments in which one or more features or aspects shown and/or described as part of a particular embodiment could be eliminated, and also encompasses additional or alternative embodiments that combine two or more features or aspects shown and/or described as part of different embodiments. Therefore, the appended claims, and not the detailed description, are intended to particularly point out subject matter regarded to be aspects of the invention, including certain but not necessarily all of the aspects and alternatives described in the detailed description.

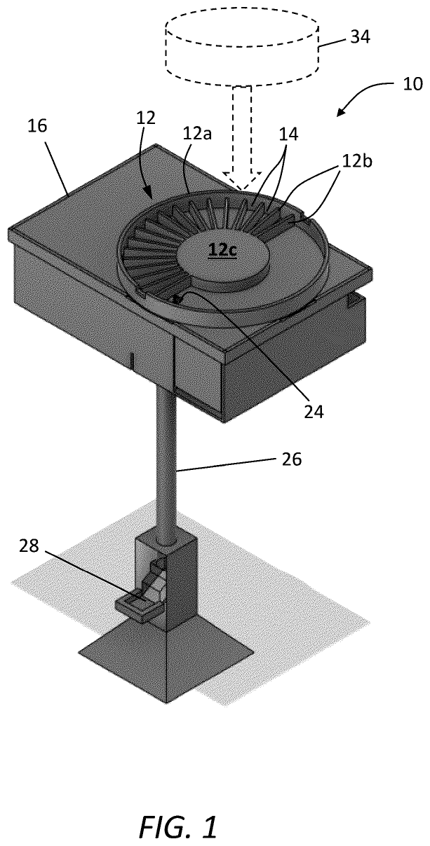

FIGS. 1 through 5 depict an automated drug delivery system 10 (also referred to herein simply as a “delivery system 10” for convenience) for delivering drug doses to animal test subjects, for example, rodents such as mice, according to certain nonlimiting aspects of the present invention. Although the invention will be described hereinafter in reference to the delivery system 10 shown in the drawings, it will be appreciated that the teachings of the invention are more generally applicable to a variety of types of applications, such as, but not limited to, delivering individual doses of food, supplements, or other materials to other types of animals, such as dogs, ferrets, guinea pigs, primates, rabbits, and/or swine. Further, while the invention will be described hereinafter in reference to use in a laboratory setting for conducting monitored drug experiments on rodents (or other animals), the invention could be used for other purposes and/or in other settings, such as for controlled delivery of food and/or medication to livestock in an agricultural setting and/or for to pets in a residential setting.

To facilitate the description provided below of the delivery system 10 represented in the drawings, relative terms, including but not limited to, “proximal,” “distal,” “anterior,” “posterior,” “vertical,” “horizontal,” “lateral,” “front,” “rear,” “side,” “forward,” “rearward,” “top,” “bottom,” “upper,” “lower,” “above,” “below,” “right,” “left,” etc., may be used in reference to the orientation of the delivery system 10 during its use and/or as represented in the drawings. All such relative terms are useful to describe the illustrated embodiment(s) but should not be otherwise interpreted as limiting the scope of the invention.

In the nonlimiting embodiment of FIGS. 1 through 5, the delivery system 10 is specifically configured and used for the controlled delivery of individual drug doses to rodents, such as laboratory mice and rats, in Max 75 cage bases during monitored drug experiments. In this particular configuration, the delivery system 10 may also be referred to as a rodent automated drug delivery system (or “RADDS”). It is understood that any description herein relative to rodents in a lab testing environment is equally applicable to other types of animals and/or other types of environments, as noted previously herein.

The automated drug delivery system 10 represented in the drawings is configured to provide drug pellets to mice based on researcher needs, allow for mouse monitoring to confirm drug delivery, enable behavioral studies to be conducted using operant condition, and/or to fit into a specific lab research animal cage. The delivery system 10 in this example is configured to fit into a Max 75 research cage base such as commercially available from Alternative Design Manufacturing & Supply, Inc, in Siloam Springs, Arkansas. Thus, the delivery system 10 has a form factor that fits into the Max 75 cage base. However, the delivery system 10 may also be configured with a form factor that fits into other sizes, shapes, and/or configurations of cages. When drug doses are delivered in a pelletized form, the pellets may combine a test product, such as a drug, with a food product in a relatively dry format to encourage the subject animal to actually ingest the pellet, and thereby also ingest the test product.

The automated drug delivery system 10 represented in the drawings may be utilized to deliver only one pellet containing a predefined drug dose at a time, though the delivery of more than one pellet at a time is also within the scope of the invention. The delivery system 10 is preferably capable of controlling the delivery of drug doses for at least three (and preferably more) days, and/or has a tray for storing several individual drug doses. The delivery system 10 may be equipped with or otherwise operate in combination with a camera configured to monitor mice that receive drug doses from the delivery system 10. The delivery system 10 may be further equipped to generate a signal that can be perceived by a test subject, such as a beep within a frequency range that is audible to a rodent, in order to train test subjects.

FIGS. 1 through 5 represent the delivery system 10 as including a carrier cartridge (tray) 12 that carries and dispenses a plurality of drug doses in the form of pellets. The pellets may include both a predefined drug dose mixed with a food product to encourage the subject animal to ingest the drug dose along with the food product. The carrier cartridge 12 includes a plurality of individual dose receptacles 14, such as for pellets, wherein each dose (pellet) receptacle 14 is shaped and sized to receive a single drug dose in the form of a pellet. In this example, the carrier cartridge 12 is in the form of a delivery barrel, which is shaped as a circular disc with an outer wall 12a and a plurality of radial walls 12b extending from a central hub 12c to the outer wall 12a. Each receptacle 14 is defined by a wedge-shaped cavity defined by and between adjacent pairs of the radial walls 12b.

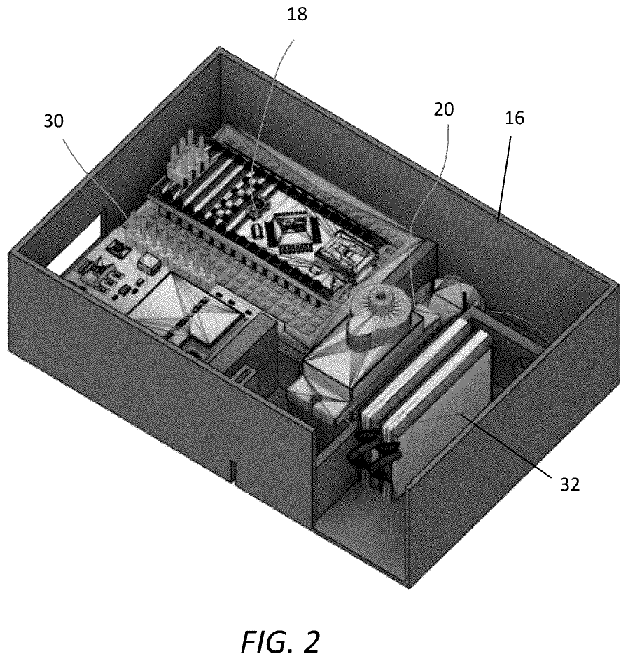

FIG. 1 depicts the carrier cartridge 12 as disposed on a top side of a main body 16 that holds various control components therein, such as depicted in FIG. 2. The main body 16 has the form factor of a rectangular box defining an interior compartment in which the control components are disposed and preferably enclosed. In this nonlimiting example, the main body 16 has the form factor of a relatively shallow rectangular tray that fits into a Max 75 cage base 36 as shown in FIG. 4. The main body 16 may have other shapes and/or sizes, for example, specifically adapted for fitting into one or more different shapes and/or sizes of cages. Preferably, all the exterior components of the delivery system 10 have rounded corner angles to reduce the risk of injury to a, animal subject test. Preferably, the delivery system 10 maintains a distance, for example, about 0.1 mm, between food, water, and air delivery systems so that it will work alongside water, air, and food delivery.

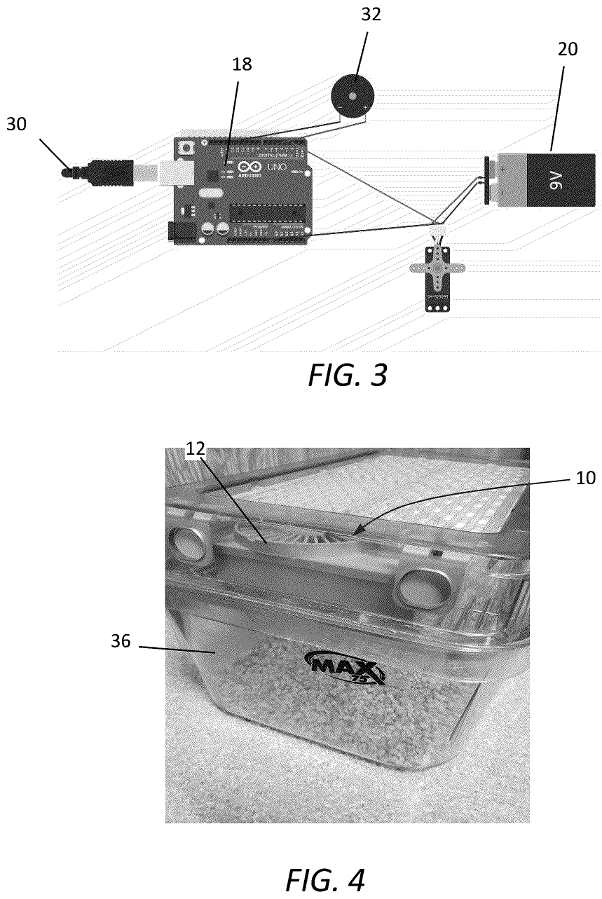

As represented in FIGS. 2 and 3, the control components disposed within the main body 16 may include a controller 18 and a power source 20. The controller 18 includes at least one and preferably more digital processors with software and/or hardware that are configured to run control programs to implement various control functions of the delivery system 10 as described herein. The power source 20 in this example is represented as a battery, preferably a rechargeable battery. However, other types of electrical power sources could be used, such as an AC-DC power converter for receiving AC power from a typical AC power outlet. Other well understood control components, such as switches, wiring, motors, and/or user interface devices, are provided in a manner well understood in the art. The user interface is configured for allowing a user to control and/or modify control programs implemented by the controller 18. The user interface may include switches, keys, a touch screen, and/or a wired or wireless data coupling that allows a user to input program instructions to the controller 18 from a remote device, such as computer or mobile device.

To dispense an individual drug dose (e.g., food pellet carrying a drug dose), the carrier cartridge 12 rotates about its hub 12c to align dose receptacles 14 with an outlet 24 in the main body 16 that opens to a passageway through the main body 16. The controller 18 preferably controls the rotation of the cartridge 12 to move in radial increments sufficient to align individual receptacles with the outlet 24, whereby each incremental movement of the cartridge 12 causes an immediately adjacent receptacle 14 to be aligned with the outlet 24. The passageway is represented as a tube 26 connected with a dispenser 28 below the main body 16 from which the test subject (e.g., rodent or other animal in the cage base 36) can access a dispensed drug dose (e.g., pellet). The passageway within the tube 26 extends downwardly from the outlet 24 so that a pellet will travel to the dispenser 28 by force of gravity. The dispenser 28 is represented as in the form of a small receiver at the bottom end of the tube 26 and sized to catch the pellet as it exits the tube 26. When the carrier cartridge 12 advances a dose receptacle 14 holding a pellet over the outlet 24, the pellet in the receptacle 14 falls into the outlet 24 and drops down through the tube 26 to the dispenser 28, where the test subject can access and eat the pellet. Preferably, the controller 18 can be easily programmed by the user such that the dispensing intervals can be selected and/or set by an operator at the controller 18 either directly at the delivery system 10 or remotely.

FIGS. 2 and 3 represent the control components as including a monitoring device 30 for monitoring whether a test subject actually takes a drug dose. In this example, the monitoring device 30 includes a camera carried in the main body 16 and aimed through an opening (not shown) in the main body 16 toward the dispenser 28 disposed beneath the main body 16, so as to be able to record one or more images of the area around the dispenser 28 when a drug dose is delivered so that the camera will record the mouse ingesting the drug dose. Other types of monitoring devices could be used, as long as they can record the test subject receiving the drug dose. The monitoring device 30 is operatively connected with and controlled by the controller 18 to obtain and/or record the data. For example, the controller 18 causes the camera to take a picture of the pellet being ingested by the rodent. Preferably, such a camera is a flashless camera to minimize any psychological stress to the test animal subject. The picture data may be stored on the monitoring device 30, the controller 18, and/or any other suitable data storage mechanism. In this way, the delivery system 10 allows the researchers to confirm that the rodent has ingested the drug pellets without requiring them to be physically present at each dosing interval.

The delivery system 10 includes a signaling device 32 configured to provide a perceptible signal to the animal subject when the carrier cartridge 12 is to dispense each drug dose in order to train the subject animal, for example to come to the dispenser 28 when a drug dose is dispensed. The signaling device 32 in this example is an audio device, such as a speaker, that generates an audible signal in a frequency that is audible to the subject animal as the drug dose is being delivered. Other types of signaling devices could be used, for example a visual signaling device such as a light that turns on or off, a tactile signaling device such as a vibrator that turns on or off, and/or combinations of audio, visual, and tactile signaling devices.

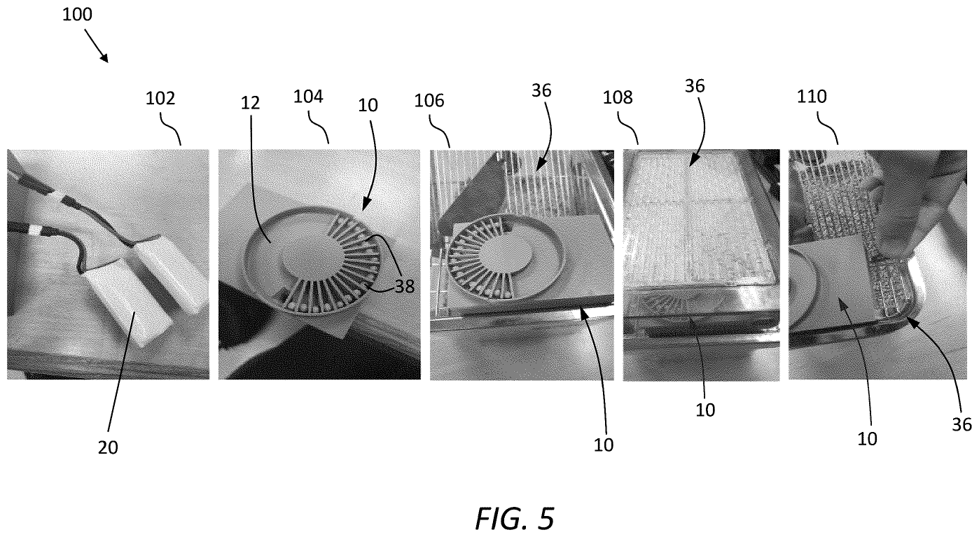

With reference to FIGS. 4 and 5, in operation, the delivery system 10 is configured to dispense at least one of the drug doses to an animal subject at preselected dispensing intervals over a selected period of time. For example, the delivery system 10 can be set up to deliver a single pellet 38 containing a drug dose every so many hours (e.g., every eight hours) for a predetermined period of time (e.g., a period of six days). The pellets 38 to be delivered are stored and/or carried within the carrier cartridge 12. Optionally, FIG. 1 represents that an additional pellet storage compartment 34 for storing additional drug dosage pellets 38 may be operatively connected with the carrier cartridge 12 to provide an extended supply of pellets 38 for a longer period of time. The controller 18 is configured by the user (e.g., a lab technician) to dispense a pellet 38 at one or more specified time intervals across the selected period of time. The user preferably makes sure that there are enough pellets 38 in the carrier cartridge 12 and/or storage compartment 34 to last for the entire period of time. The dispensing intervals may be all the same time period, such as every eight hours until the end of the experiment, or may be different time periods, such as every four hours during the day and then an eight-hour interval at night. The loaded and programmed delivery system 10 is assembled and placed into the cage base 36 (e.g., Max 75 cage base) with a test animal subject (e.g., lab mouse). In one nonlimiting example method 100, as illustrated in FIG. 5, at 102 the power source(s) (e.g., battery) 20 are plugged into the delivery system 10. Then at 104, the pellets 38 are placed into the delivery barrel (carrier cartridge 12). Next at 106, the delivery system 10 is placed in the cage base 36 (with the test animal subject) and turned on, and, after waiting the period of time for the experiment to be over at 108, at 110 the monitoring data (e.g., pictures on a micro SD) can be collected and viewed to verify whether the animal subject ingested each drug dose in accordance with the applicable test requirements.

In some nonlimiting arrangements, the automated drug delivery system 10 may optionally include Wi-Fi capabilities and video streaming capabilities, for example, to allow for real time monitoring of the monitoring device and the animal subject remotely by researchers.

As previously noted above, though the foregoing detailed description describes certain aspects of one or more particular embodiments of the invention, alternatives could be adopted by one skilled in the art. For example, the automated drug delivery system 10 and its components could differ in appearance and construction from the embodiments described herein and shown in the drawings, functions of certain components of the automated drug delivery system 10 could be performed by components of different construction but capable of a similar (though not necessarily equivalent) function, and various materials could be used in the fabrication of the automated drug delivery system 10 and/or its components. As such, and again as was previously noted, it should be understood that the invention is not necessarily limited to any particular embodiment described herein or illustrated in the drawings.

1. An automated drug delivery system comprising:
a carrier cartridge configured to carry and dispense a plurality of drug doses;
a monitoring device; and
a controller operatively coupled with the carrier cartridge and the monitoring device,
wherein the controller is configured to operate the carrier cartridge to dispense at least one of the plurality of drug doses to an animal subject at each of a plurality of preselected dispensing intervals, and
wherein the controller is configured to operate the monitoring device to record whether an animal subject ingests each drug dose dispensed by the carrier cartridge.

2. The automated drug delivery system of claim 1, wherein the controller comprises one or more digital processors implementing programming to operate the carrier cartridge and to operate the monitoring device.

3. The automated drug delivery system of claim 1, wherein the monitoring device comprises a camera configured to obtain an image of the animal subject when the carrier cartridge dispenses each drug dose.

4. The automated drug delivery system of claim 1, further comprising:
a signaling device configured to provide a perceptible signal to the animal subject when the carrier cartridge is to dispense each drug dose.

5. The automated drug delivery system of claim 4, wherein the signaling device emits an audio signal in a frequency range audible by the animal subject.

6. The automated drug delivery system of claim 4, wherein the signaling device comprises an audio speaker.

7. The automated drug delivery system of claim 1, wherein each dispensing interval is the same time period.

8. The automated drug delivery system of claim 1, wherein the at least some of the dispensing intervals are different time periods.

9. The automated drug delivery system of claim 1, wherein the dispensing intervals can be selected and/or set by an operator at the controller.

10. The automated drug delivery system of claim 1, wherein the carrier cartridge, monitoring device, and controller are configured to dispense the drug doses and monitor the subjects for a period of three days or more.

11. The automated drug delivery system of claim 1, further comprising an electrical power source configured to provide electrical power to the carrier cartridge, the monitoring device, the controller, and/or the signaling device.

12. The automated drug delivery system of claim 11, wherein the power source comprises a battery.

13. The automated drug delivery system of claim 1, wherein the animal subject is a rodent.

14. The automated drug delivery system of claim 1, further comprising a dispenser from which the animal subject can access a dispensed drug dose, wherein the dispenser is operatively connected with the carrier cartridge such that a drug dose dispensed by the carrier cartridge is accessible to the animal subject at the dispenser.

15. The automated drug delivery system of claim 14, further comprising a passageway that operatively connects the carrier cartridge with the dispenser to direct the dispensed drug dose to the dispenser.

16. The automated drug delivery system of claim 1, wherein the carrier cartridge is configured to dispense only one drug dosing unit at each interval.

17. The automated drug delivery system of claim 16, wherein the drug dosing unit is in the form of a food pellet.

18. The automated drug delivery system of claim 1, wherein the automated drug delivery system is provided as a single unit having a form factor that is sized and shaped to fit into a laboratory rodent cage.

19. A method of automatically delivering a drug dose to an animal subject using an automated drug delivery system comprising a carrier cartridge configured to carry and dispense a plurality of drug doses, a monitoring device, and a controller operatively coupled with the carrier cartridge and the monitoring device, the method comprising:
operating the carrier cartridge with the controller to dispense at least one of the plurality of drug doses to the animal subject at each of a plurality of preselected dispensing intervals; and
operating the monitoring device with the controller to record whether the animal subject ingests each drug dose dispensed by the carrier cartridge.

20. The method of claim 19, further comprising generating a signal with a signaling device of the automated drug delivery system when the carrier cartridge is to dispense each drug dose, wherein the signal is perceptible to the animal subject.
